(19) United States
(12) Patent Application Publication
(10) Pub. No.: US 2024/0180491 A1
(43) Pub. Date: Jun. 6, 2024

(54) Cut-to-Length Sensing Catheters and Methods Thereof

(71) Applicant: Bard Access Systems, Inc., Salt Lake City, UT (US)

(72) Inventors: Vivek Pandey, Ujjain (IN); Leandro Forciniti, Wharton, NJ (US); Chetan S. Dhuri, Bangalore (IN)

(21) Appl. No.: 18/074,078

(22) Filed: Dec. 2, 2022

(51) Int. Cl. A61B 5/00; A61B 5/01

(52) U.S. Cl. CPC A61B 5/6852; A61B 5/01

(57) ABSTRACT

Cut-to-length sensing catheters retain their sensing capabilities and methods thereof. For example, a sensing catheter can include a catheter tube having temperature sensors along an initial length thereof. Each temperature sensor is independently electronically addressed, thereby enabling a temperature-sensing capability of the sensing catheter to be maintained despite excising any one or more temperature sensors with a distal length of the catheter tube upon cutting the catheter tube to a working length. Such a sensing catheter can further include strain sensors respectively paired with the temperature sensors along the initial length of the catheter tube, thereby enabling correction of temperature-measurement uncertainty in any temperature sensor by way of a local strain measurement. Such a sensing catheter can further include lactate sensors respectively paired with the temperature sensors, thereby enabling enzyme activity and, thus, lactate concentration, associated with any lactate sensor to be normalized by way of at least local-temperature compensation.

### BACKGROUND

Catheters such as central venous catheters (“CVCs”), peripherally inserted central catheters (“PICCs”), midline catheters, and peripheral intravenous catheters (“PIVCs”) are indwelling medical devices for delivery of medication, sampling of blood, and the like. Being that the foregoing catheters are indwelling medical devices, they have potential for sensing and transmitting electrical signals associated with different physiological parameters of clinical interest. However, such catheters, particularly PICCs, are cut to length prior to placement in patients. This makes it difficult to incorporate sensors for the sensing in locations on the foregoing catheters often best suited for the sensing. Indeed, the sensing in a PICC is often best in a distal-end portion of the PICC in view of proximity to a heart of a patient, but it is the distal-end portion of the PICC that is cut when cutting the PICC to length prior to placement in the patient. What is needed are sensing catheters with well-placed sensors that can be cut to length without compromising their sensing capabilities.

Disclosed herein are cut-to-length sensing catheters and methods thereof that address the foregoing.

### SUMMARY

Disclosed herein is a cut-to-length temperature-sensing catheter including, in some embodiments, a catheter hub, a catheter tube including a plurality of temperature sensors, and one or more extension legs operably connected together. The catheter tube has a proximal-end portion inserted into a bore of a distal portion of the catheter hub. The plurality of temperature sensors is disposed in or on a surface of the catheter tube along an initial length thereof. Each temperature sensor of the plurality of temperature sensors is independently electronically addressed with corresponding temperature-sensor electrical leads leading thereto. This enables a temperature-sensing capability of the sensing catheter to be maintained despite excising any one or more temperature sensors of the plurality of temperature sensors with a distal length of the catheter tube upon cutting the catheter tube from the initial length to a working length. Each extension leg of the one-or-more extension legs has a distal-end portion inserted into a proximal portion of the catheter hub.

In some embodiments, the plurality of temperature sensors is a plurality of nested thermocouples.

In some embodiments, each thermocouple of the plurality of thermocouples includes a longitudinal loop formed between two conducting lines of dissimilar thermocouple conductors having distal portions disposed in or on the surface of the catheter tube. The two conducting lines distally terminate in a hot junction in or on the surface of the catheter tube.

In some embodiments, the two conducting lines have proximal portions disposed in or on an outer surface of the catheter hub. The two conducting lines proximally terminate in a cold junction on a printed circuit board assembly of the catheter hub.

In some embodiments, the proximal portions of the two conducting lines extend from the outer surface of the catheter hub into the bore of the catheter hub. The proximal portions of the two conducting lines within the bore of the hub form an electrical junction with the distal portions of the two conducting lines where the proximal-end portion of the catheter tube is inserted into the bore of the catheter hub.

In some embodiments, the thermocouple conductors of the two conducting lines are conducting polymers.

In some embodiments, the plurality of temperature sensors is a plurality of resistance temperatures detectors (“RTDs”).

In some embodiments, each RTD of the plurality of RTDs includes a temperature-sensing element of an RTD conductor formed in or on the surface of the catheter tube with a known temperature vs. resistance relationship. This enables any measured electrical resistance across the temperature-sensing element to be converted to a temperature.

In some embodiments, the RTD conductor is nanoscale-structured silver or gold.

In some embodiments, the sensing catheter further includes a plurality of strain sensors disposed in or on the surface of the catheter tube along the initial length thereof. Each strain sensor of the plurality of strain sensors is independently electronically addressed with corresponding strain-sensor electrical leads leading thereto. The enables a strain-sensing capability of the sensing catheter to be maintained despite excising any one or more strain sensors of the plurality of strain sensors with the distal length of the catheter tube upon cutting the catheter tube from the initial length to the working length.

In some embodiments, the plurality of strain sensors and the plurality of temperature sensors are respectively paired along the initial length of the catheter tube. This enables temperature-measurement uncertainty resulting from strain-induced inhomogeneity in any temperature sensor of the plurality of temperature sensors to be corrected by way of a local strain measurement.

In some embodiments, each strain sensor of the plurality of strain sensors includes a patterned strain-sensitive element of a strain-sensor conductor formed in or on the surface of the catheter tube with a length along that of the catheter tube. This enables any change in electrical resistance resulting from resistance-increasing tension or resistance-decreasing compression across the patterned strain-sensitive element induced by bending the catheter tube to be measured.

In some embodiments, the strain-sensor conductor is nanoscale-structured silver or gold.

In some embodiments, the surface of the catheter tube independently includes an abluminal surface or a luminal surface of the catheter tube.

In some embodiments, the sensing catheter further includes an electrical connector. The electrical connector is configured to connect a console with sensor electronics of the sensing catheter and relay electrical signals to the sensing catheter, relay electrical signals from the sensing catheter, or both.

In some embodiments, the sensing catheter is a CVC, a PICC, a midline catheter, or PIVC.

Disclosed herein is a cut-to-length lactate-sensing catheter including, in some embodiments, a catheter hub, a catheter tube including a plurality of lactate sensors, and one or more extension legs operably connected together. The catheter tube has a proximal-end portion inserted into a bore of a distal portion of the catheter hub. The plurality of lactate sensors is disposed in or on a surface of the catheter tube along an initial length thereof. Each lactate sensor of the plurality of lactate sensors is independently electronically addressed with corresponding lactate-sensor electrical leads leading thereto. This enables a lactate-sensing capability of the sensing catheter to be maintained despite excising any one or more lactate sensors of the plurality of lactate sensors with a distal length of the catheter tube upon cutting the catheter tube from the initial length to a working length. Each extension leg of the one-or-more extension legs has a distal-end portion inserted into a proximal portion of the catheter hub.

In some embodiments, each lactate sensor of the plurality of lactate sensors includes either a three-electrode sensor or a two-electrode sensor. The three-electrode sensor includes a working electrode, a reference electrode, and a counter electrode. The two-electrode sensor includes the working electrode and the reference electrode.

In some embodiments, each lactate sensor of the plurality of lactate sensors includes the two-electrode sensor with an antifouling membrane thereover. The working electrode includes a layered working-electrode structure of a metal layer under a conducting-polymer layer having an immobilized enzyme therein or thereon. The reference electrode includes a reference-electrode structure of a same or different metal layer as the working electrode under a metal-salt layer.

In some embodiments, the sensing catheter further includes a plurality of temperature sensors disposed in or on the surface of the catheter tube along the initial length thereof. Each temperature sensor of the plurality of temperature sensors is independently electronically addressed with corresponding temperature-sensor electrical leads leading thereto. This enables a temperature-sensing capability of the sensing catheter to be maintained despite excising any one or more temperature sensors of the plurality of temperature sensors with the distal length of the catheter tube upon cutting the catheter tube from the initial length to a working length.

In some embodiments, the plurality of temperature sensors and the plurality of lactate sensors are respectively paired along the initial length of the catheter tube. This enables enzyme activity and, thus, lactate concentration, associated with any lactate sensor of the plurality of lactate sensors to be normalized by way of at least local-temperature compensation.

In some embodiments, the plurality of temperature sensors is a plurality of nested thermocouples.

In some embodiments, each thermocouple of the plurality of thermocouples includes a longitudinal loop formed between two conducting lines of dissimilar thermocouple conductors having distal portions disposed in or on the surface of the catheter tube. The two conducting lines distally terminate in a hot junction in or on the surface of the catheter tube.

In some embodiments, the two conducting lines have proximal portions disposed in or on an outer surface of the catheter hub. The two conducting lines proximally terminate in a cold junction on a printed circuit board assembly of the catheter hub.

In some embodiments, the proximal portions of the two conducting lines extend from the outer surface of the catheter hub into the bore of the catheter hub. The proximal portions of the two conducting lines within the bore of the hub form an electrical junction with the distal portions of the two conducting lines where the proximal-end portion of the catheter tube is inserted into the bore of the catheter hub.

In some embodiments, the thermocouple conductors of the two conducting lines are conducting polymers.

In some embodiments, the plurality of temperature sensors is a plurality of RTDs.

In some embodiments, each RTD of the plurality of RTDs includes a temperature-sensing element of an RTD conductor formed in or on the surface of the catheter tube with a known temperature vs. resistance relationship. This enables any measured electrical resistance across the temperature-sensing element to be converted to a temperature.

In some embodiments, the RTD conductor is nanoscale-structured silver or gold.

In some embodiments, the sensing catheter further includes a plurality of strain sensors disposed in or on the surface of the catheter tube along the initial length thereof. Each strain sensor of the plurality of strain sensors is independently electronically addressed with corresponding strain-sensor electrical leads leading thereto. The enables a strain-sensing capability of the sensing catheter to be maintained despite excising any one or more strain sensors of the plurality of strain sensors with the distal length of the catheter tube upon cutting the catheter tube from the initial length to the working length.

In some embodiments, the plurality of strain sensors and the plurality of temperature sensors are respectively paired along the initial length of the catheter tube. This enables temperature-measurement uncertainty resulting from strain-induced inhomogeneity in any temperature sensor of the plurality of temperature sensors to be corrected by way of a local strain measurement.

In some embodiments, each strain sensor of the plurality of strain sensors includes a patterned strain-sensitive element of a strain-sensor conductor formed in or on the surface of the catheter tube with a length along that of the catheter tube. This enables any change in electrical resistance resulting from resistance-increasing tension or resistance-decreasing compression across the patterned strain-sensitive element induced by bending the catheter tube to be measured.

In some embodiments, the strain-sensor conductor is nanoscale-structured silver or gold.

In some embodiments, the surface of the catheter tube independently includes an abluminal surface or a luminal surface of the catheter tube.

In some embodiments, the sensing catheter further includes an electrical connector. The electrical connector is configured to connect a console with sensor electronics of the sensing catheter and relay electrical signals to the sensing catheter, relay electrical signals from the sensing catheter, or both.

In some embodiments, the sensing catheter is a CVC, a PICC, a midline catheter, or a PIVC.

Also disclosed herein is a method of using a sensing catheter including, in some embodiments, an excising step, a catheter tube-advancing step, and sensor data-reading step. The excising step includes excising a distal length of a catheter tube from an initial length thereof to cut the catheter tube down to a working length. The excising of the distal length of the catheter tube also excises one or more sensors disposed in or on a surface of the catheter tube. The catheter tube-advancing step includes advancing a distal portion of the catheter tube into a blood-vessel lumen of a patient to a desired location within the patient. The sensor data-reading step includes reading sensor data from the one-or-more sensors to determine an instantaneous condition of the patient.

In some embodiments, the catheter tube includes a plurality of lactate sensors disposed in or on the surface of the catheter tube along the initial length thereof. Each lactate sensor of the plurality of lactate sensors is independently electronically addressed with corresponding lactate-sensor electrical leads leading thereto. This enables a lactate-sensing capability of the sensing catheter to be maintained despite the excising of the one-or-more sensors with the distal length of the catheter tube.

In some embodiments, the catheter tube further includes a plurality of temperature sensors disposed in or on the surface of the catheter tube along the initial length thereof. Each temperature sensor of the plurality of temperature sensors is independently electronically addressed with corresponding temperature-sensor electrical leads leading thereto. This enables a temperature-sensing capability of the sensing catheter to be maintained despite the excising of the one-or-more sensors with the distal length of the catheter tube.

In some embodiments, the plurality of temperature sensors and the plurality of lactate sensors are respectively paired along the initial length of the catheter tube. This enables enzyme activity and, thus, lactate concentration, associated with any lactate sensor of the plurality of lactate sensors to be normalized by way of at least local-temperature compensation.

In some embodiments, the plurality of temperature sensors is a plurality of nested thermocouples.

In some embodiments, the plurality of temperature sensors is a plurality of RTDs.

In some embodiments, the catheter tube further includes a plurality of strain sensors disposed in or on the surface of the catheter tube along the initial length thereof. Each strain sensor of the plurality of strain sensors is independently electronically addressed with corresponding strain-sensor electrical leads leading thereto. This enables a strain-sensing capability of the sensing catheter to be maintained despite the excising of the one-or-more sensors with the distal length of the catheter tube.

In some embodiments, the plurality of strain sensors and the plurality of temperature sensors are respectively paired along the initial length of the catheter tube. This enables temperature-measurement uncertainty resulting from strain-induced inhomogeneity in any temperature sensor of the plurality of temperature sensors to be corrected by way of a local strain measurement.

In some embodiments, the method further includes an electrical connector-connecting step. The electrical connector-connecting step includes connecting an electrical connector of the sensing catheter to a console for the reading of the sensor data from a display screen associated with the console. The electrical connector is configured to connect a console with sensor electronics of the sensing catheter and relay electrical signals to the sensing catheter, relay electrical signals from the sensing catheter, or both.

These and other features of the concepts provided herein will become more apparent to those of skill in the art in view of the accompanying drawings and following description, which describe particular embodiments of such concepts in greater detail.

### DRAWINGS

FIG. 1 illustrates a catheter-sensing system including a console and a cut-to-length sensing catheter in accordance with some embodiments.

FIG. 2A illustrates a detailed view of a first side of the sensing catheter, particularly a first side of a catheter tube of the sensing catheter, which side of the catheter tube includes both a plurality of temperature sensors and a plurality of strain sensors in accordance with some embodiments.

FIG. 2B illustrates a detailed view of a second side of the sensing catheter, particularly a second side of the catheter tube of the sensing catheter, which side of the catheter tube includes a plurality of lactate sensors in accordance with some embodiments.

FIG. 3A illustrates a more detailed view of the first side of the catheter tube including a temperature sensor of the plurality of temperature sensors and a strain sensor of the plurality of strain sensors in accordance with some embodiments.

FIG. 3B illustrates a more detailed view of the second side of the catheter tube including a lactate sensor of the plurality of lactate sensors in accordance with some embodiments.

FIG. 4 illustrates a detailed view of the first side of the sensing catheter including an alternative plurality of temperature sensors in accordance with some embodiments.

FIG. 5 illustrates a cutaway view of a catheter hub of the sensing catheter of FIG. 4 including proximal portions of conducting lines disposed in or on an outer surface of the catheter hub extending into a bore of the catheter hub in accordance with some embodiments.

FIG. 6 illustrates a detailed view of a catheter tube including distal portions of the conducting lines disposed in or on an abluminal surface of the catheter tube being inserted into the catheter hub of FIG. 5, thereby forming an electrical junction with the proximal portions of conducting lines extending into the bore of the catheter hub in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” refers to a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device, and, in such instances, the proximal portion, the proximal section, or the proximal length of the medical device can be further specified as a “proximal-end portion,” a “proximal-end section,” or a “proximal-end length” of the medical device if not otherwise suggested.

“Distal” refers to a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device, and, in such instances, the distal portion, the distal section, or the distal length of the medical device can be further specified as a “distal-end portion,” a “distal-end section,” or a “distal-end length” of the medical device if not otherwise suggested.

Unless defined otherwise, all technical and scientific terms used herein have the same meaning as commonly understood by those of ordinary skill in the art.

Catheters such as CVCs, PICCs, midline catheters, and PIVCs are indwelling medical devices for delivery of medication, sampling of blood, and the like. Being that the foregoing catheters are indwelling medical devices, they have potential for sensing and transmitting electrical signals associated with different physiological parameters of clinical interest. However, such catheters, particularly PICCs, are cut to length prior to placement in patients. This makes it difficult to incorporate sensors for the sensing in locations on the foregoing catheters often best suited for the sensing. Indeed, the sensing in a PICC is often best in a distal-end portion of the PICC in view of proximity to a heart of a patient, but it is the distal-end portion of the PICC that is cut when cutting the PICC to length prior to placement in the patient. What is needed are sensing catheters with well-placed sensors that can be cut to length without compromising their sensing capabilities.

Disclosed herein are cut-to-length sensing catheters and methods thereof that address the foregoing.

### Sensing Systems

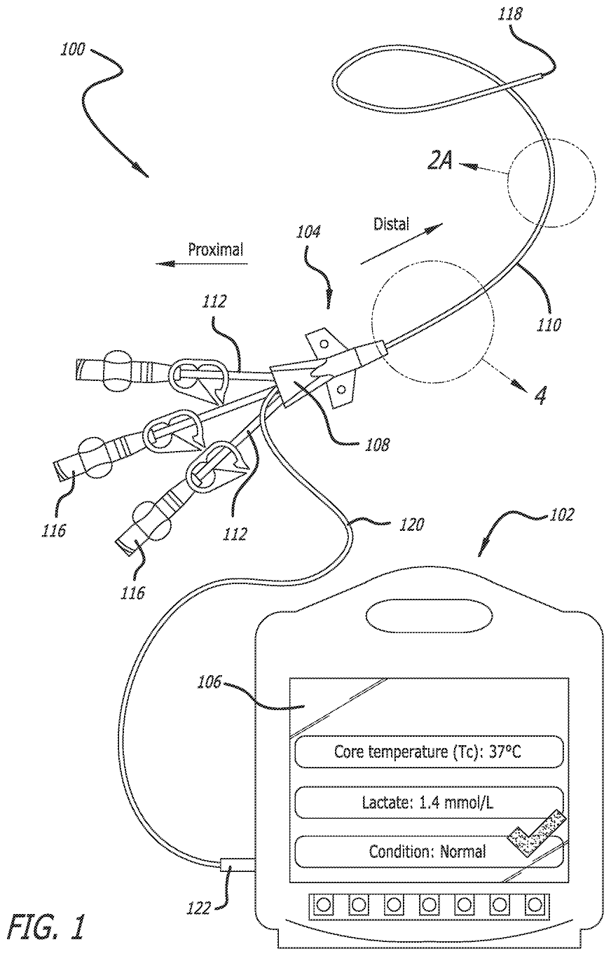

FIG. 1 illustrates a catheter-sensing system 100 including a console 102 and a cut-to-length sensing catheter 104 in accordance with some embodiments.

As shown, the console 102 can include a display screen 106 integrated therein; however, the display screen 106 can alternatively be separated from the console 102 and communicatively coupled thereto. Such a display screen 106 can be configured to provide a graphical user interface (“GUI”) and display information thereon such as sensor data from the sensing catheter 104 as well as patient condition based upon the sensor data.

While not shown, the console 102 can include one or more processors and memory including instructions stored in the memory configured to instantiate one or more processes when executed by the one-or-more processors for controlling various functions of the system 100, optionally, with specialized logic therefor.

The one-or-more processes can be configured to relay electrical signals from the console 102 to the sensing catheter 104, relay electrical signals from the sensing catheter 104 to the console 102, or both. In an example, the one-or-more processes can be configured to apply, with respect to the reference electrode 134, a voltage to the working electrode 132 of any lactate sensor of the plurality of lactate sensors 124 set forth below, read a current from the working electrode 132 of the foregoing lactate sensor, or both. In another example, the one-or-more processes can be configured to send a higher-voltage current to any RTD of the plurality of RTDs 148 set forth below, receive a lower-voltage current from any RTD of the plurality of RTDs 148, or both. In another example, the one-or-more processes can be configured to apply a higher voltage to any strain sensor of the plurality of strain sensors 128 set forth below, read a lower voltage from any strain sensor of the plurality of strain sensors 128, or both. In another example, the one-or-more processes can be configured to read a voltage from any thermocouple of the plurality of thermocouples 150.

The one-or-more processes can additionally or alternatively be configured to automatically determine sensor measurements from the electrical signals relayed from the sensing catheter 104 to the console 102. In an example, a lactate concentration can be automatically and amperometrically determined using a current read from the working electrode 132 of any lactate sensor of the plurality of lactate sensors 124, which current is proportional to the lactate concentration. In another example, a temperature can be automatically determined using a voltage drop read across any RTD of the plurality of RTDs 148, which voltage drop corresponds to an electrical resistance, and which electrical resistance, in turn, corresponds to the temperature in view of a known temperature vs. resistance relationship of the RTD conductor.

Lastly, the one-or-more processes can additionally or alternatively be configured to compile the sensor data from the sensor measurements, optionally, for determining trends in the sensor data; determine an instantaneous condition of a patient from the sensor data; and display the sensor data, the instantaneous condition of the patient, or both on the display screen 106 as shown in FIG. 1.

While not shown, the console 102 can further include an electrical cable having an electrical connector configured to connect the console 102 to an external power supply. Additionally or alternatively, the console 102 can include an internal power supply (e.g., a battery). Whether the console 102 is configured for the external power supply, the internal power supply, or both, the console 102 can include power management circuitry for power regulation and distribution therein as well as to the sensing catheter 104.

Notably, the sensing catheter 104, which is set forth below, can include the extension cable 120 and at least the proximal electrical connector 122 thereof. The extension cable 120 can be configured to connect the sensor electronics of the sensing catheter 104 to the console 102 for relaying the electrical signals from the console 102 to the sensing catheter 104, relaying the electrical signals from the sensing catheter 104 to the console 102, or both. The extension cable 120 can also be also configured to connect the sensing catheter 104 to the console 102 for powering the sensing catheter 104. That said, the extension cable 120 can be alternatively configured to connect the sensing catheter 104 to the external power supply set forth below, and the electrical signals relayed from the console 102 to the sensing catheter 104 as well as those from the sensing catheter 104 to the console 102 can be relayed wirelessly. Additionally or alternatively, the sensing catheter 104 can include the internal power supply set forth below, and the electrical signals relayed from the console 102 to the sensing catheter 104 as well as those from the sensing catheter 104 to the console 102 can be relayed wirelessly.

Notwithstanding the foregoing, the system 100 need not include the console 102 because the sensing catheter 104, itself, can be configured with a visualizing means for visualizing the sensor data, the patient condition based upon the sensor data, or both; an embedded system for controlling various functions of the system 100 or sensing catheter 104; power circuitry; an internal power supply (e.g., a battery), the extension cable 120 configured to connect the sensing catheter 104 to the external power supply, or both; or a combination thereof. For example, the visualizing means of such a sensing catheter 104 can include an integrated display screen (e.g., a twisted-nematic thin film-transistor liquid-crystal display [“TN TFT LCD” ] display screen); one or more light-emitting diodes (“LEDs”) configured to emit light in one or more colors, one or more patterns (e.g., a simple flashing pattern, a more complex flashing pattern with at least two different flashes such as shorter and longer flashes, etc.), or a combination thereof; or both the integrated display screen and the one-or-more LEDs for indicating the sensor data, the patient condition based upon the sensor data, or both. Further to the example, the embedded system of the sensing catheter 104 can include a catheter computer module (“CCM”) having a combination of one or more processors, memory, and instructions stored in the memory configured to instantiate one or more processes like those set forth above when executed by the one-or-more processors for controlling various functions of the system 100 or sensing catheter 104, optionally, with specialized logic therefor. Lastly to the example, the sensing catheter 104 can include the internal battery supply or battery.

### Cut-to-Length Sensing Catheters

FIG. 1 illustrates a full view of the cut-to-length sensing catheter 104 in accordance with some embodiments.

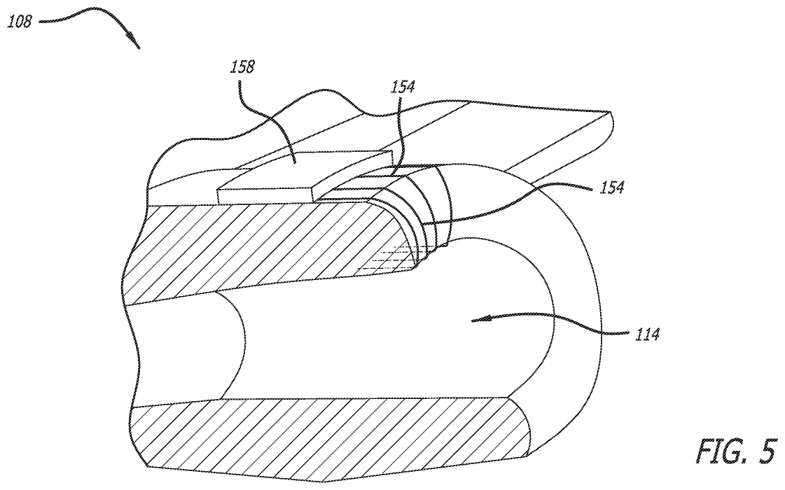

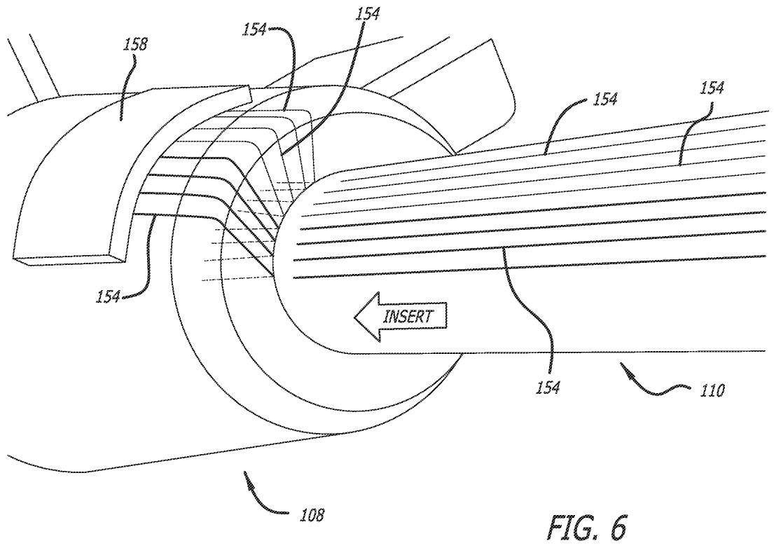

As shown, the sensing catheter 104 can include a catheter hub 108, a catheter tube 110, and one or more extension legs 112 operably connected together. Indeed, the catheter tube 110 can have a proximal-end portion inserted into a bore 114 of a distal portion of the catheter hub 108. (See FIGS. 5 and 6 for more detail.) Such a catheter tube 110 can be formed of silicone, a thermoplastic urethane, a thermoplastic elastomer, or the like. Further, the catheter tube 110 can have a coating thereover such as a hydrophilic coating, a heparin coating, or a nitrous oxide-releasing coating. When the catheter tube 110 is coated with the hydrophilic coating, the hydrophilic coating can be polyvinylpyrrolidone (“PVP”), poly(2-hydroxyethyl methacrylate) (“pHEMA”), or polyethylene glycol (“PEG”) at a thickness of 1-50 μm, optionally, functionalized with heparin or an antimicrobial agent such as silver, chlorhexidine, or one or more antimicrobial peptides. Each extension leg of the one-or-more extension legs 112 can have a distal-end portion inserted into a proximal portion of the catheter hub 108 as well as a proximal-end portion inserted into a distal portion of an extension-leg connector 116 (e.g., a Luer connector). Such a sensing catheter 104 can be monoluminal or multiluminal with at least one lumen 117 extending from a proximal opening in an extension-leg connector to a distal opening in a catheter tip 118 of the catheter tube 110. Any other lumen can extend from another proximal opening in another extension-leg connector to another distal opening in a side of the catheter tube 110.

Notably, the sensing catheter 104 having one or more lumens such as the lumen 117 establishes at least two surfaces of the sensing catheter 104, namely an inner surface and an outer surface of the sensing catheter 104. The inner surface and the outer surface can alternatively be referred to herein as a luminal surface and an abluminal surface, respectively, particularly with respect to the catheter tube 110 or any extension leg of the one-or-more extension legs 112. Any surface referenced herein without further reference to it being the inner or luminal surface or the outer or abluminal surface should be understood as independently being either the inner or luminal surface or the outer or abluminal surface unless context suggests otherwise. However, it should be understood that the one-or-more pluralities of sensors set forth below being on the outer or abluminal surface of at least the catheter tube 110 confers an advantage over them being on the inner or luminal surface of the catheter tube 110 in that the one-or-more pluralities of sensors on the outer or abluminal surface of the catheter tube 110 can make direct contact with a sample (e.g., blood) including an analyte (e.g., lactate), obviate or at least mitigate temperature effects from lower-temperature infusates delivered through the one-or-more lumens, or the like.

The sensing catheter 104 can also include an extension cable 120 having a proximal electrical connector 122 and, optionally, a distal electrical connector configured to connect the extension cable 120 to the catheter hub 108 if the extension cable 120 is not fixedly connected thereto. The extension cable 120 can be configured to connect sensor electronics of the sensing catheter 104 to the console 102 by way of the proximal electrical connector 122 for relaying the electrical signals from the console 102 to the sensing catheter 104, relaying the electrical signals from the sensing catheter 104 to the console 102, or both. The extension cable 120 can also be configured to connect the sensing catheter 104 to the console 102 by way of the proximal electrical connector 122 for powering the sensing catheter 104. In some embodiments, however, the extension cable 120 can be configured to connect the sensing catheter 104 to the external power supply by way of the proximal electrical connector 122. In such embodiments including the console 102, the electrical signals relayed from the console 102 to the sensing catheter 104 as well as those from the sensing catheter 104 to the console 102 can be relayed wirelessly.

While the sensing catheter 104 of FIG. 1 is a PICC, it should be understood the sensing catheter 104 can alternatively be a CVC, a midline catheter, a PIVC, or any other vascular access device (“VAD”) ranging from at least 1.4 to 10 French, if applicable to the VAD, that can benefit from the features of the sensing catheter 104 provided herein. Another embodiment of the sensing catheter 104 can even include a urinary catheter such as a Foley catheter; however, a Foley catheter can be limited to single instances of the sensors provided herein in view of its inverted configuration to the sensing catheter 104 provided herein. Notably, neither the form nor the stiffness of the sensing catheter 104 is appreciably altered from that of its counterpart, non-sensing catheter by the one-or-more pluralities of the sensors set forth below. As such, clinicians can easily substitute a non-sensing catheter with the sensing catheter 104 of a same kind.

For expository expediency, an embodiment of the sensing catheter 104 will now be described having three pluralities of individually electronically addressed sensors, wherein each plurality of the three pluralities of sensors corresponds to a different sensing capability and, thus, three different sensing capabilities of the sensing catheter 104. As will become more apparent in view of the following description, each sensor of each plurality of the three pluralities of sensors being individually electronically addressed enables the three different sensing capabilities of the sensing catheter 104 to be maintained despite excising any one or more sensors of the three pluralities of sensors with a distal length of the catheter tube 110 upon cutting the catheter tube 110 from an initial length to a working length. And it should be understood maintenance of such sensing capabilities of the sensing catheter 104 upon cutting the catheter tube 110 extend to each embodiment of the sensing catheter 104 provided herein, whether the sensing catheter 104 is a temperature-sensing catheter including only the plurality of temperature sensors 126 or a multiple analyte-sensing catheter including one or more pluralities of other analyte sensors in addition to the plurality of lactate sensors 124.

The embodiment of the sensing catheter 104 set forth below, which is, namely, a lactate-sensing catheter with an additional sensing capability for at least core body temperature Tc, has a plurality of lactate sensors 124, a plurality of temperature sensors 126, and a plurality of strain sensors 128 in a synergistic combination for the three pluralities of sensors. Indeed, as will become more apparent in view of the following description, temperature-measurement uncertainty resulting from catheter-bending, strain-induced inhomogeneity in any temperature sensor of the plurality of temperature sensors 126 can be corrected by way of a local strain measurement with a corresponding strain sensor. Likewise, enzyme activity and, thus, lactate concentration, associated with any lactate sensor of the plurality of lactate sensors 124 can be normalized by way of at least local-temperature compensation with a corrected temperature measurement from a corresponding temperature sensor. And, of course, in view of the above, each sensor of the plurality of lactate sensors 124, the plurality of temperature sensors 126, and the plurality of strain sensors 128 being individually electronically addressed enables the lactate-, temperature-, and strain-sensing capabilities of the sensing catheter 104 and their synergy to be maintained despite excising any one or more sensors of the foregoing pluralities of sensors with a distal length of the catheter tube 110 upon cutting the catheter tube 110 from the initial length to a working length.

Lastly, it should be understood that the sensing catheter 104 is not limited to the lactate-sensing catheter set forth below. Indeed, a plurality of other metabolite (e.g., glucose, creatinine, etc.) sensors or, more generally, a plurality of other analyte sensors in addition to the plurality of lactate sensors 124 or as an alternative thereto can likewise benefit from being associated with the plurality of temperature sensors 126 and the plurality of strain sensors 128 in the sensing catheter 104. That said, the sensing catheter 104 can include, in some embodiments, one or more of the foregoing pluralities of analyte sensors (e.g. the plurality of lactate sensors 124) without the plurality of strain sensors 128 and, further, without even the plurality of temperature sensors 126 despite the synergistic combination illustrated by way of the plurality of lactate sensors 124, the plurality of temperature sensors 126, and the plurality of strain sensors 128. Such embodiments of the sensing catheter 104 can be less expensive to produce and, thus, offered at a lower price, albeit with potential for lowered measurement accuracy. The sensing catheter 104 need not even include one or more of the foregoing pluralities of analyte sensors (e.g. the plurality of lactate sensors 124) in some embodiments. Indeed, the sensing catheter 104 can instead solely be a temperature-sensing catheter for Tc with or without the plurality of strain sensors 128, again, with the understanding that omitting the plurality of strain sensors 128 can make the sensing catheter 104 less expensive to produce but with potential for lowered measurement accuracy.

Notably, about 1.7 million people in the United States of America are diagnosed with sepsis every year, and, of those people, about 270,000 die. Lactate is a major biomarker for sepsis and, for this reason, has a significant role in detecting, monitoring, and treating sepsis. Currently, blood samples are collected every 4-8 hours for measuring biomarkers such as lactate in hospitalized patients at risk of sepsis. For if a case of sepsis in a patient is detected early and treated immediately, mortality rate can improve by 7.6% every hour. Therefore, continuous monitoring of lactate, particularly, continuous in vivo monitoring of lactate, using the sensing catheter 104 or another VAD configured in kind can make a significant difference in clinical outcomes. That, and continuous monitoring of Tc, particularly, continuous in vivo monitoring of Tc, using the sensing catheter 104 or another VAD configured in kind can further make a significant difference in clinical outcomes as changes in body temperature also have a significant role in detecting, monitoring, and treating adverse conditions.

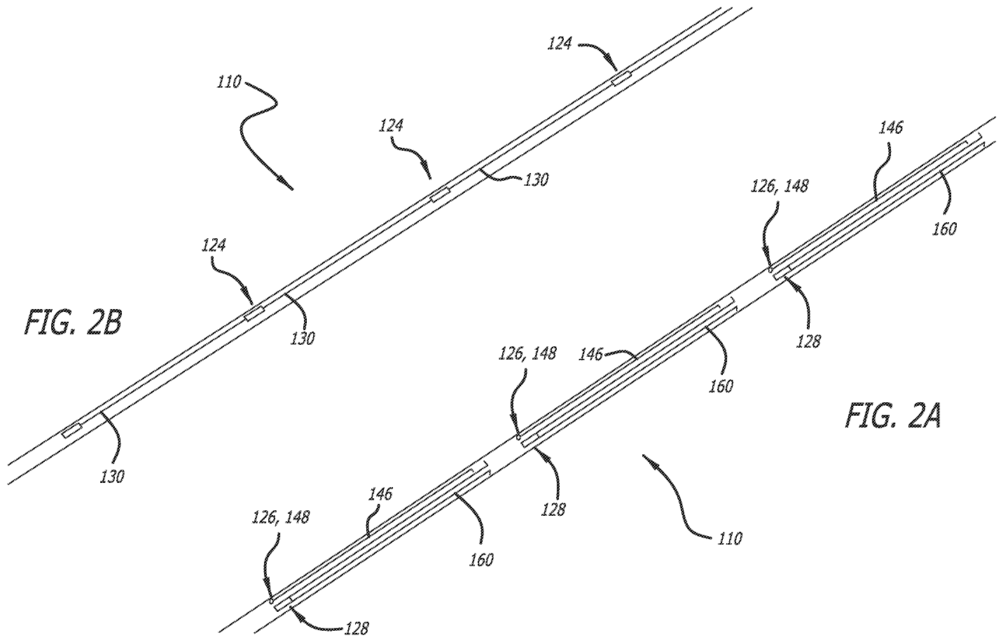

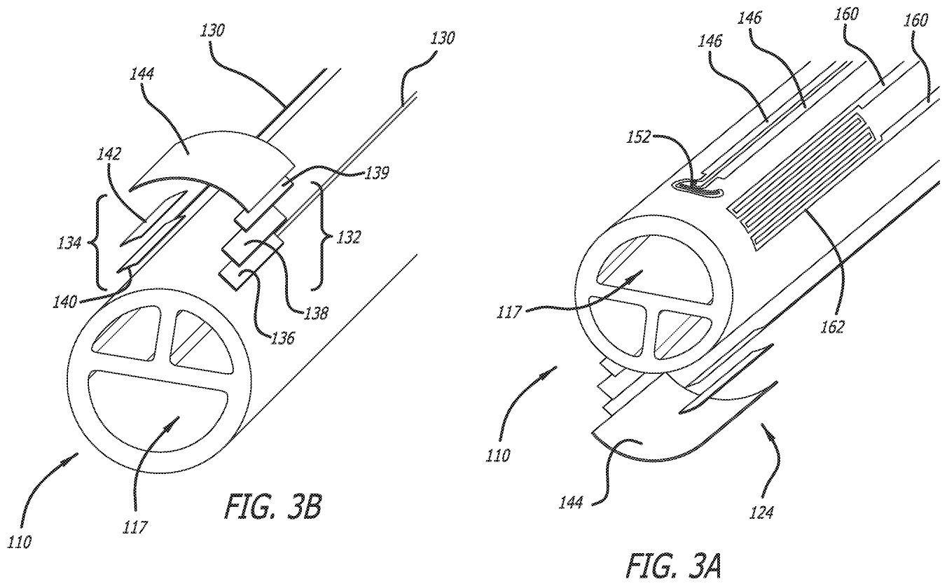

FIGS. 2A and 3A illustrate detailed views of a first side of the sensing catheter 104, particularly a first side of the catheter tube 110, which side of the catheter tube 110 includes the plurality of temperature sensors 126 and the plurality of strain sensors 128 in accordance with some embodiments. FIGS. 2B and 3B illustrate detailed views of a second side of the sensing catheter 104 opposite the first side thereof, particularly a second side of the catheter tube 110, which side of the catheter tube 110 includes a plurality of lactate sensors 124 in accordance with some embodiments. As such, the sensing catheter 104 can include three pluralities of sensors, wherein each plurality of the three pluralities of sensors corresponds to a different sensing capability and, thus, a total of three different sensing capabilities of the sensing catheter 104. However, it should be understood that the three pluralities of sensors can be distributed over the catheter tube 110 differently than that shown in the foregoing figures. For example, three-sensor sets, each set of which can include a single lactate sensor, a single temperature sensor, and a single strain sensor in-line, can be distributed along the catheter tube 110 such that they circumscribe a spiral around the catheter with a period coextensive with the initial length of the catheter tube 110, a fraction of the initial length of the catheter tube 110, or a multiple of the initial length of the catheter tube 110.

As shown, the plurality of lactate sensors 124 can be disposed in or on a surface of the catheter tube 110 along the initial length thereof. Each lactate sensor of the plurality of lactate sensors 124 is independently electronically addressed with corresponding lactate-sensor electrical leads 130 leading thereto. This enables a lactate-sensing capability of the sensing catheter 104 to be maintained despite excising any one or more lactate sensors 124 of the plurality of lactate sensors 124 with a distal length of the catheter tube 110 upon cutting the catheter tube 110 from the initial length to a working length.

Each lactate sensor of the plurality of lactate sensors 124 can include, but is not limited to, a three-electrode or two-electrode lactate sensor at, for example, a microscale or smaller. For example, each electrode of the three-electrode or two-electrode sensor can range from a few microns in thickness to a few hundred microns in length or width. Each lactate sensor of the three-electrode and two-electrode sensor can include a working electrode 132 and a reference electrode 134; however, the three-electrode lactate sensor can further include a counter electrode. The working electrode 132 of at least the two-electrode lactate sensor can include a layered working-electrode structure of a metal layer 136 (e.g., silver, gold, or chromium) having, for example, a nanoscale thickness (e.g., a thickness of a few nanometers) under a conducting-polymer layer 138 (e.g., polypyrrole, poly(3,4-ethylenedioxythiophene) (“PEDOT”), PEDOT:polystyrene sulfonate (“PSS”) [i.e., PEDOT:PSS], PEDOT:Cl, etc.) also having, for example, a nanoscale thickness. The conducting-polymer layer 138 includes an immobilized enzyme (e.g., lactate oxidase [“LOD” ] or lactate dehydrogenase [“LDH” ]) disposed in the conducting-polymer layer 138 or on a surface of the conducting-polymer layer 138 as shown by enzyme layer 139. The reference electrode 134 of at least the two-electrode lactate sensor can include a layered reference-electrode structure of a same or different metal layer 140 as the working electrode 132 (e.g., the same metal such as silver) under a metal-salt layer 142 (e.g., silver chloride), each of the metal layer 140 and the metal-salt layer 142 also having, for example, a nanoscale thickness. Whether each lactate sensor of the plurality of lactate sensors 124 includes the three-electrode lactate sensor or the two-electrode lactate sensor, each lactate sensor of the plurality of lactate sensors 124 can include an antifouling membrane 144 thereover. The antifouling membrane 144 can be a thin (e.g., ≤1 μm), porous membrane configured to allow lactate molecules therethrough but simultaneously block biomolecules such as proteins, thereby allowing the sensing catheter 104 and the plurality of lactate sensor thereof to operate in blood without fouling.

As further shown in FIGS. 2A, 2B, 3A, and 3B, the plurality of temperature sensors 126 can also be disposed in or on the surface of the catheter tube 110 along the initial length thereof. Each temperature sensor of the plurality of temperature sensors 126 is independently electronically addressed with corresponding temperature-sensor electrical leads 146 leading thereto or the two conducting lines 154 of dissimilar thermocouple conductors. This enables a temperature-sensing capability of the sensing catheter 104 to be maintained despite excising any one or more temperature sensors 126 of the plurality of temperature sensors 126 with a distal length of the catheter tube 110 upon cutting the catheter tube 110 from the initial length to a working length. Notably, the plurality of temperature sensors 126 and the plurality of lactate sensors 124 are respectively paired along the initial length of the catheter tube 110. This enables enzyme activity and, thus, lactate concentration, associated with any lactate sensor of the plurality of lactate sensors 124 to be normalized by way of at least local-temperature compensation as enzyme activity increases with temperature.

The plurality of temperature sensors 126 can be a plurality of RTDs 148 or a plurality of nested thermocouples 150.

Beginning with the RTDs, which are shown in FIGS. 2A and 3A, each RTD of the plurality of RTDs 148 can include a temperature-sensing element 152 (e.g., a planar coil having a diameter ≤100 μm) of an RTD conductor formed in or on the surface of the catheter tube 110. The temperature-sensing element 152 is specifically paired with a proximate lactate sensor of the plurality of lactate sensors 124. The RTD conductor of the temperature-sensing element 152 has a known temperature vs. resistance relationship. This enables any measured electrical resistance across the temperature-sensing element 152 to be converted to a temperature. Notably, the RTD conductor can be nanoscale-structured silver (e.g., silver nanowire, silver nanoparticles, etc.) or gold (e.g., gold nanowire, gold nanoparticles, etc.) disposed, deposited (e.g., via electrochemical deposition, chemical vapor deposition, or physical vapor deposition), coated (e.g., via spray coating or spin coating), printed (e.g., via screen printing), or the like in or on the surface of the catheter tube 110, but the RTD conductor is not limited thereto. Indeed, the RTD conductor can alternatively be a thin film of platinum, nickel, or copper.

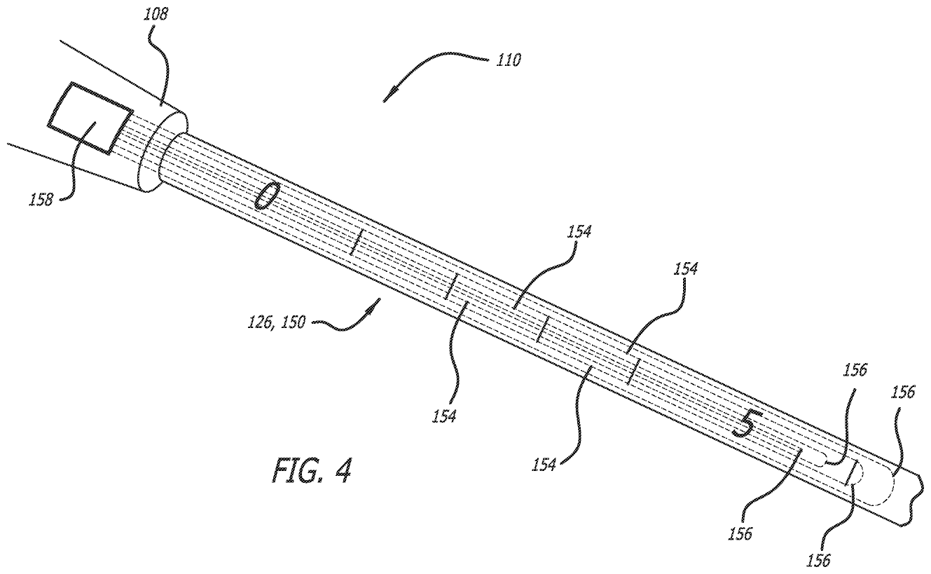

FIG. 4 illustrates a detailed view of the first side of the sensing catheter 104 including the plurality of thermocouples 150 in accordance with some embodiments. It should be understood, however, the plurality of thermocouples 150 can be greater in number and extend farther along the catheter tube 110 than that shown in FIG. 4.

Continuing with the thermocouples shown in FIG. 4, each thermocouple of the plurality of thermocouples 150 can include a longitudinal loop formed between two conducting lines 154 of dissimilar thermocouple conductors disposed, deposited (e.g., via electrochemical deposition, chemical vapor deposition, or physical vapor deposition), coated (e.g., via spray coating or spin coating), printed (e.g., via screen printing), or the like in or on at least the surface of the catheter tube 110 having, for example, a microscale thickness (e.g., ≤10 μm) and a nanoscale-to-microscale width (e.g., a width of a few hundred nanometers to a few hundred micrometers). Not including an outermost thermocouple, each thermocouple of the plurality of thermocouples 150 can be disposed within an outer thermocouple, thereby forming the plurality of nested thermocouples referenced above. The two conducting lines 154 forming the longitudinal loop of each thermocouple of the plurality of thermocouples 150 distally terminate, for example, with overlapping distal ends, in a hot junction 156 in or on the surface of the catheter tube 110. The hot junction 156 is specifically paired with a proximate lactate sensor of the plurality of lactate sensors 124. The two conducting lines 154 also proximally terminate in a cold junction (not shown) on a printed circuit board assembly 158 of the catheter hub 108. Notably, the thermocouple conductors of the two conducting lines 154 can be differently doped conducting polymers such as a p-type conducting polymer and an n-type conducting polymer with relatively high electrical conductivities (e.g., a few S/cm) and relatively low thermal conductivities (e.g., a few W/[m·K]). Such conducting polymers can be PEDOT:Cl and polyaniline (“PANI”), respectively.

FIG. 5 illustrates a cutaway view of the catheter hub 108 of the sensing catheter 104 including proximal portions of the conducting lines 154 disposed in or on the outer surface of the catheter hub 108 in accordance with some embodiments. FIG. 6 illustrates a detailed view of the catheter tube 110 including distal portions of the conducting lines 154 disposed in or on the abluminal surface of the catheter tube 110 in accordance with some embodiments.

As shown, the two conducting lines 154 can have proximal portions disposed in or on the outer surface of the catheter hub 108. Further, the proximal portions of the two conducting lines 154 can extend from the outer surface of the catheter hub 108, around a distal face of the catheter hub 108, and into the bore 114 of the catheter hub 108 for forming an electrical junction. The two conducting lines 154 can also have distal portions disposed in or on the abluminal surface of the catheter tube 110. The proximal portions of the two conducting lines 154 within the bore 114 of the hub form the electrical junction with the distal portions of the two conducting lines 154 in or on the abluminal surface of the catheter tube 110 when the proximal-end portion of the catheter tube 110 is inserted into the bore 114 of the catheter hub 108. FIG. 5 illustrates forming such an electrical junction as the proximal-end portion of the catheter tube 110 is inserted into the bore 114 of the catheter hub 108.

As further shown in FIGS. 2A, 2B, 3A, and 3B, the plurality of strain sensors 128 can also be disposed in or on the surface of the catheter tube 110 along the initial length thereof. Each strain sensor of the plurality of strain sensors 128 is independently electronically addressed with corresponding strain-sensor electrical leads 160 leading thereto. The enables a strain-sensing capability of the sensing catheter 104 to be maintained despite excising any one or more strain sensors 128 of the plurality of strain sensors 128 with a distal length of the catheter tube 110 upon cutting the catheter tube 110 from the initial length to a working length. Notably, the plurality of strain sensors 128 and the plurality of temperature sensors 126 are respectively paired along the initial length of the catheter tube 110. This enables temperature-measurement uncertainty resulting from strain-induced inhomogeneity in any temperature sensor of the plurality of temperature sensors 126 to be corrected by way of a local strain measurement. Additionally or alternatively, an adverse event such as an occlusion in a blood vessel or extravasation therefrom can be detected as a local strain measurement in a proximate strain sensor of the plurality of strain sensors 128.

Each strain sensor of the plurality of strain sensors 128 can include a patterned strain-sensitive element 162 (e.g., a serpentine structure having a length up to a few millimeters and a width ≤100 μm) of a strain-sensor conductor formed in or on the surface of the catheter tube 110 with a length along that of the catheter tube 110. The patterned strain-sensitive element 162 is specifically paired with the temperature-sensing element 152 of a proximate RTD of the plurality of RTDs 148 or the hot junction 156 of a proximate thermocouple of the plurality of thermocouples 150. This enables any change in electrical resistance resulting from resistance-increasing tension or resistance-decreasing compression across the patterned strain-sensitive element 162 induced by bending the catheter tube 110 to be measured. Notably, the strain-sensor conductor is nanoscale-structured silver or gold, optionally, matching that of the RTD conductor.

### Methods

Methods include methods of using the sensing catheter 104 and methods of the system 100 or the sensing catheter 104 itself. For example, a method of using the sensing catheter 104 can include one or more steps selected from a needle tract-establishing step, an access guidewire-advancing step, an introducer needle-withdrawing step, an excising step, a first catheter-advancing step, a guidewire-exchanging step, a second catheter-advancing step, a maneuver guidewire-withdrawing step, an electrical connector-connecting step, and sensor data-reading step. Notably, the foregoing method is for the sensing catheter 104, which is configured as a PICC; however, the method can be adapted as needed for CVCs, midline catheters, PIVCs, or the like.

The needle tract-establishing step can include establishing a needle tract from an area of skin to a blood-vessel lumen of a patient with an introducer needle.

The access guidewire-advancing step can include advancing a distal portion of an access guidewire into the blood-vessel lumen through the introducer needled until access to the blood-vessel lumen is secured with the access guidewire.

The introducer needle-withdrawing step can include withdrawing the introducer needle from the blood-vessel lumen over the access guidewire and leaving the access guidewire in place in the blood-vessel lumen.

The excising step can include excising a distal length of the catheter tube 110 from the initial length thereof to cut the catheter tube 110 down to a working length. The excising of the distal length of the catheter tube 110 can also excise one or more sensors disposed in or on the surface of the catheter tube 110; however, as set forth above, the one-or-more sensors possibly excised with the distal length of the catheter tube 110 are individually electronically addressed such that the sensing capabilities of the sensing catheter 104 are maintained despite their possible excision with the distal length of the catheter tube 110. Notably, the one-or-more sensors possibly excised with the distal length of the catheter tube 110 can be any sensor of the synergistic combination of the plurality of lactate sensors 124, the plurality of temperature sensors 126, and the plurality of strain sensors 128 in some embodiments. However, the sensing catheter 104 can have any of a number of different configurations with respect to its one or more pluralities of sensors as set forth above, any of which can be encompassed by the instant method.

The first catheter-advancing step can include advancing a distal portion of the catheter tube 110 over the access guidewire into the blood-vessel lumen until access to the blood-vessel lumen is secured with the catheter tube 110.

The guidewire-exchanging step can include exchanging the access guidewire with a maneuver guidewire in a combination of an access guidewire-withdrawing step and a maneuver guidewire-advancing step. The access guidewire-withdrawing step can include withdrawing the access guidewire from the blood-vessel lumen through the lumen 117 of the sensing catheter 104 in which it is disposed while keeping the catheter tube 110 in place in the blood-vessel lumen. The maneuver guidewire-advancing step can include advancing the maneuver guidewire into the blood-vessel lumen through the foregoing lumen 117 of the sensing catheter 104 until a distal end of the maneuver guidewire is in a desired location with the patient such as a lower ⅓ of a superior vena cava (“SVC”) of the patient.

The second catheter-advancing step can include advancing the catheter tube 110 farther into the blood-vessel lumen over the maneuver guidewire until the catheter tip 118 is advanced to the desired location (e.g., the lower ⅓ of the SVC) within the patient.

The maneuver guidewire-withdrawing step can include withdrawing the maneuver guidewire from the blood-vessel lumen through the lumen 117 of the sensing catheter 104 in which it is disposed while keeping the catheter tube 110 in place in the desired location within the patient.

The electrical connector-connecting step can include connecting the proximal electrical connector 122 of the sensing catheter 104 to the console 102 for the reading of the sensor data from the display screen 106 associated with the console 102. As set forth above, the proximal electrical connector 122 is configured to connect the console 102 with the sensor electronics of the sensing catheter 104 and relay the electrical signals to the sensing catheter 104, relay the electrical signals from the sensing catheter 104, or both.

The sensor data-reading step can include reading the sensor data from the one-or-more sensors to determine an instantaneous condition of the patient. Such sensor data can be displayed on the display screen 106 associated with the console 102. And, while a clinician can determine the instantaneous condition of the patient from the sensor data in accordance with the foregoing, the console 102 can also automatically determine the instantaneous condition of the patient and display it on the display screen 106 for the clinician in some embodiments.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. A cut-to-length temperature-sensing catheter, comprising:
a catheter hub;
a catheter tube having a proximal-end portion inserted into a bore of a distal portion of the catheter hub;
a plurality of temperature sensors disposed in or on a surface of the catheter tube along an initial length thereof, each temperature sensor of the plurality of temperature sensors independently electronically addressed with corresponding temperature-sensor electrical leads leading thereto, thereby enabling a temperature-sensing capability of the sensing catheter to be maintained despite excising any one or more temperature sensors of the plurality of temperature sensors with a distal length of the catheter tube upon cutting the catheter tube from the initial length to a working length; and
one or more extension legs, each extension leg of the one-or-more extension legs having a distal-end portion inserted into a proximal portion of the catheter hub.

2. The sensing catheter of claim 1, wherein the plurality of temperature sensors is a plurality of nested thermocouples.

3. The sensing catheter of claim 2, wherein each thermocouple of the plurality of thermocouples includes a longitudinal loop formed between two conducting lines of dissimilar thermocouple conductors having distal portions disposed in or on the surface of the catheter tube, the two conducting lines distally terminating in a hot junction in or on the surface of the catheter tube.

4. The sensing catheter of claim 3, wherein the two conducting lines have proximal portions disposed in or on an outer surface of the catheter hub, the two conducting lines proximally terminating in a cold junction on a printed circuit board assembly of the catheter hub.

5. The sensing catheter of claim 4, wherein the proximal portions of the two conducting lines extend from the outer surface of the catheter hub into the bore of the catheter hub, the proximal portions of the two conducting lines within the bore of the hub forming an electrical junction with the distal portions of the two conducting lines where the proximal-end portion of the catheter tube is inserted into the bore of the catheter hub.

6. The sensing catheter of claim 3, wherein the thermocouple conductors of the two conducting lines are conducting polymers.

7. The sensing catheter of claim 1, wherein the plurality of temperature sensors is a plurality of resistance temperatures detectors (“RTDs”).

8. The sensing catheter of claim 7, wherein each RTD of the plurality of RTDs includes a temperature-sensing element of an RTD conductor formed in or on the surface of the catheter tube with a known temperature vs. resistance relationship, thereby enabling any measured electrical resistance across the temperature-sensing element to be converted to a temperature.

9. The sensing catheter of claim 7, wherein the RTD conductor is nanoscale-structured silver or gold.

10. The sensing catheter of claim 1, further comprising a plurality of strain sensors disposed in or on the surface of the catheter tube along the initial length thereof, each strain sensor of the plurality of strain sensors independently electronically addressed with corresponding strain-sensor electrical leads leading thereto, thereby enabling a strain-sensing capability of the sensing catheter to be maintained despite excising any one or more strain sensors of the plurality of strain sensors with the distal length of the catheter tube upon cutting the catheter tube from the initial length to the working length.

11. The sensing catheter of claim 10, wherein the plurality of strain sensors and the plurality of temperature sensors are respectively paired along the initial length of the catheter tube, thereby enabling temperature-measurement uncertainty resulting from strain-induced inhomogeneity in any temperature sensor of the plurality of temperature sensors to be corrected by way of a local strain measurement.

12. The sensing catheter of claim 10, wherein each strain sensor of the plurality of strain sensors includes a patterned strain-sensitive element of a strain-sensor conductor formed in or on the surface of the catheter tube with a length along that of the catheter tube, thereby enabling any change in electrical resistance resulting from resistance-increasing tension or resistance-decreasing compression across the patterned strain-sensitive element induced by bending the catheter tube to be measured.

13. The sensing catheter of claim 12, wherein the strain-sensor conductor is nanoscale-structured silver or gold.

14. The sensing catheter of claim 1, wherein the surface of the catheter tube independently includes an abluminal surface or a luminal surface of the catheter tube.

15. The sensing catheter of claim 1, further comprising:
an electrical connector configured to connect a console with sensor electronics of the sensing catheter and relay electrical signals to the sensing catheter, relay electrical signals from the sensing catheter, or both.

16. The sensing catheter of claim 1, wherein the sensing catheter is a central venous catheter (“CVC”), a peripherally inserted central catheter (“PICC”), a midline catheter, or a peripheral intravenous catheter (“PIVC”).

17. A cut-to-length lactate-sensing catheter, comprising:
a catheter hub;
a catheter tube having a proximal-end portion inserted into a bore of a distal portion of the catheter hub;
a plurality of lactate sensors disposed in or on a surface of the catheter tube along an initial length thereof, each lactate sensor of the plurality of lactate sensors independently electronically addressed with corresponding lactate-sensor electrical leads leading thereto, thereby enabling a lactate-sensing capability of the sensing catheter to be maintained despite excising any one or more lactate sensors of the plurality of lactate sensors with a distal length of the catheter tube upon cutting the catheter tube from the initial length to a working length; and
one or more extension legs, each extension leg of the one-or-more extension legs having a distal-end portion inserted into a proximal portion of the catheter hub.

18. The sensing catheter of claim 17, wherein each lactate sensor of the plurality of lactate sensors includes either a three-electrode sensor or a two-electrode sensor, the three-electrode sensor including a working electrode, a reference electrode, and a counter electrode, and the two-electrode sensor including the working electrode and the reference electrode.

19. The sensing catheter of claim 18, wherein each lactate sensor of the plurality of lactate sensors includes the two-electrode sensor with an antifouling membrane thereover, the working electrode including a layered working-electrode structure of a metal layer under a conducting-polymer layer having an immobilized enzyme therein or thereon, and the reference electrode including a layered reference-electrode structure of a same or different metal layer as the working electrode under a metal-salt layer.

20. The sensing catheter of claim 17, further comprising a plurality of temperature sensors disposed in or on the surface of the catheter tube along the initial length thereof, each temperature sensor of the plurality of temperature sensors independently electronically addressed with corresponding temperature-sensor electrical leads leading thereto, thereby enabling a temperature-sensing capability of the sensing catheter to be maintained despite excising any one or more temperature sensors of the plurality of temperature sensors with the distal length of the catheter tube upon cutting the catheter tube from the initial length to a working length.

21. The sensing catheter of claim 20, wherein the plurality of temperature sensors and the plurality of lactate sensors are respectively paired along the initial length of the catheter tube, thereby enabling enzyme activity and, thus, lactate concentration, associated with any lactate sensor of the plurality of lactate sensors to be normalized by way of at least local-temperature compensation.

22. The sensing catheter of claim 21, wherein the plurality of temperature sensors is a plurality of nested thermocouples.

23. The sensing catheter of claim 22, wherein each thermocouple of the plurality of thermocouples includes a longitudinal loop formed between two conducting lines of dissimilar thermocouple conductors having distal portions disposed in or on the surface of the catheter tube, the two conducting lines distally terminating in a hot junction in or on the surface of the catheter tube.

24. The sensing catheter of claim 23, wherein the two conducting lines have proximal portions disposed in or on an outer surface of the catheter hub, the two conducting lines proximally terminating in a cold junction on a printed circuit board assembly of the catheter hub.

25. The sensing catheter of claim 24, wherein the proximal portions of the two conducting lines extend from the outer surface of the catheter hub into the bore of the catheter hub, the proximal portions of the two conducting lines within the bore of the hub forming an electrical junction with the distal portions of the two conducting lines where the proximal-end portion of the catheter tube is inserted into the bore of the catheter hub.

26. The sensing catheter of claim 22, wherein the thermocouple conductors of the two conducting lines are conducting polymers.

27. The sensing catheter of claim 17, wherein the plurality of temperature sensors is a plurality of resistance temperatures detectors (“RTDs”).

28. The sensing catheter of claim 27, wherein each RTD of the plurality of RTDs includes a temperature-sensing element of an RTD conductor formed in or on the surface of the catheter tube with a known temperature vs. resistance relationship, thereby enabling any measured electrical resistance across the temperature-sensing element to be converted to a temperature.

29. The sensing catheter of claim 27, wherein the RTD conductor is nanoscale-structured silver or gold.

30. The sensing catheter of claim 17, further comprising a plurality of strain sensors disposed in or on the surface of the catheter tube along the initial length thereof, each strain sensor of the plurality of strain sensors independently electronically addressed with corresponding strain-sensor electrical leads leading thereto, thereby enabling a strain-sensing capability of the sensing catheter to be maintained despite excising any one or more strain sensors of the plurality of strain sensors with the distal length of the catheter tube upon cutting the catheter tube from the initial length to the working length.

31. The sensing catheter of claim 30, wherein the plurality of strain sensors and the plurality of temperature sensors are respectively paired along the initial length of the catheter tube, thereby enabling temperature-measurement uncertainty resulting from strain-induced inhomogeneity in any temperature sensor of the plurality of temperature sensors to be corrected by way of a local strain measurement.

32. The sensing catheter of claim 30, wherein each strain sensor of the plurality of strain sensors includes a patterned strain-sensitive element of a strain-sensor conductor formed in or on the surface of the catheter tube with a length along that of the catheter tube, thereby enabling any change in electrical resistance resulting from resistance-increasing tension or resistance-decreasing compression across the patterned strain-sensitive element induced by bending the catheter tube to be measured.

33. The sensing catheter of claim 32, wherein the strain-sensor conductor is nanoscale-structured silver or gold.

34. The sensing catheter of claim 17, wherein the surface of the catheter tube independently includes an abluminal surface or a luminal surface of the catheter tube.

35. The sensing catheter of claim 17, further comprising an electrical connector configured to connect a console with sensor electronics of the sensing catheter and relay electrical signals to the sensing catheter, relay electrical signals from the sensing catheter, or both.

36. The sensing catheter of claim 17, wherein the sensing catheter is a central venous catheter (“CVC”), a peripherally inserted central catheter (“PICC”), a midline catheter, or a peripheral intravenous catheter (“PIVC”).

37-45. (canceled)
